(19) United States
(12) Patent Application Publication
(10) Pub. No.: US 2002/0001810 A1
(43) Pub. Date: Jan. 3, 2002

(54) Q-beta replicase based assays; the use of chimeric DNA-RNA molecules as probes from which efficient Q-beta replicase templates can be generated in a reverse transcriptase dependent manner

(76) Inventor: Michael Patrick Farrell, Sugar Grove, IL (US)

Correspondence Address: Michael P. Farrell, 1937 FAYS LANE, Sugar Grove, IL, 60554, US

(21) Appl. No.: 09/874,850

(22) Filed: Jun. 5, 2001

Related U.S. Application Data
(60) Provisional application No. 60/209,351, filed on Jun. 5, 2000.

(51) Int. Cl. C12Q 1/68; C07H 21/02; C07H 21/04

(52) U.S. Cl. 435/6; 536/23.1

(57) ABSTRACT

Certain small RNA molecules can serve as templates for exponential replication by Q-beta replicase. A single molecule can give rise to easily detectable replication products. This permits their detection at the single molecule level. In this patent application tripartite chimeric molecules composed of an RNA segment bounded on each side by a DNA segment are described. Although these chimeras are not templates for exponential amplification by Q-beta replicase they can give rise to such templates by enzymatic reactions which depend on the activity of reverse transcriptase. They can therefore be used as the basis for ultra-sensitive assays for reverse transcriptase. Modifications of the templates and the assays permit application of these chimeras and Q-beta replicase to ultra-sensitive nucleic acid hybridization assays and to assays for non-nucleic acid targets.

### CROSS-REFERENCE TO RELATED APPLICATIONS

This patent application is based on the provisional patent application No. 60/209,351 filed on 06/05/2000 by Michael P. Farrell of Sugar Grove, Ill.

REFERENCES

U.S. PATENT DOCUMENTS

4,786,600 November 1998 Kramer et al

4,988,617 January 1991 Landegren et al

5,112,734 May 1992 Kramer et al

5,399,491 March 1995 Kacian et al

5,407,798 April 1995 Martinelli et al

5,616,478 April 1997 Chetvarin et al

5,837,466 November 1998 Lane et al

5,763,171 June 1998 Stefano J. E.

Other Publications

Tyagi S, Landegren U, Tazi M, Lizardi P M, Kramer F R. Extremely sensitive, background-free gene detection using binary probes and beta replicase. Proc Natl Acad Sci USA May 28, 1996;93(11):5395-400

Lomeli H, Tyagi S, Pritchard C G, Lizardi P M, Kramer F R. Quantitative assays based on the use of replicatable hybridization probes. Clin Chem September 1989;35(9):1826-31

Pritchard C G, Stefano J E. Replicatable RNA reporters. Amplified detection of viral nucleic acid at subattomole levels using Q beta replicase. Ann Biol Clin (Paris) 1990;48(7):492-7

Munishkin A V, Voronin L A, Ugarov V I, Bondareva L A, Chetverina H V, Chetverin A B. Efficient templates for Q beta replicase are formed by recombination from heterologous Mol sequences. J Biol September 1991 20;221(2):463-72

Kramer F R, Lizardi P M. Replicatable RNA reporters. Nature June 1989 1;339(6223):401-2

Chetverin A B, Spirin A S. Replicable RNA vectors: prospects for cell-free gene amplification, expression, and cloning. Prog Nucleic Acid Res Mol Biol 1995;51:225-70

Burg J L, Juffras A M, Wu Y, Blomquist C L, Du Y. Mol Cell Probes 1996 Aug;10(4):257-71 Single molecule detection of RNA reporter probes by amplification with Q beta replicase.

Lomeli H, Tyagi S, Pritchard C G, Lizardi P M, Kramer F R. Quantitative assays based on the use of replicatable hybridization probes. Clin Chem September 1989 35(9):1826-31

Brown D, Gold L. Biochemistry November 1995 14;34(45):14775-82 Selection and characterization of RNAs replicated by Q beta replicase.

Rohde N, Daum H, Biebricher C K. The mutant distribution of an RNA species replicated by Q beta replicase. J Mol Biol 1995 Jun 16;249(4):754-62

Moody M D, Burg J L, DiFrancesco R, Lovem D, Stanick W, Lin-Goerke J, Mahdavi K, Wu Y, Farrell M P. Evolution of host cell RNA into efficient template RNA by Q beta replicase: the origin of RNA in untemplated reactions Biochemistry 1994 Nov 22;33(46):13836-47

Tuerk C, Gold L. Science Agust 1990 3;249(4968):505-10 Systematic evolution of ligands by exponential enrichment: RNA ligands to bacteriophage T4 DNA polymerase.

Brown D, Gold L. Template recognition by an RNA-dependent RNA polymerase: identification and characterization of two RNA binding sites on Q beta replicase. Biochemistry November 1995 14;34(45):14765-74

Blok HJ, Kramer F R. Amplifiable hybridization probes containing a molecular switch. Mol Cell Probes Jun 1997; 1l(3):187-194

Thompson J D, Decker S, Haines D, Collins RS, Feild M, Gillespie D. Enzymatic amplification of RNA purified from crude cell lysate by reversible target capture. Clin Chem September 1989 35(9):1878-81

Chen H, Brown D, Gold L. Novel methods of generating specific oligonucleotide inhibitors of viral polymerases. Methods Enzymol 1996;275:503-20

Biebricher C K. Replication and evolution of short-chained RNA species replicated by Q beta replicase. Cold Spring Harb Symp Quant Biol 1987;52:299-306

Miele E A, Mills D R, Kramer F R. Autocatalytic replication of a recombinant RNA. J Mol Biol December 1983 15;171(3):281-95

Hanes J. and Pluckthun A. In vitro selection and evolution of functional proteins by using ribosome display. Proc. Nat. Acad. Sci. USA 1997 Vol 94 pp. 4937-4942

Roberts R. W. and Szostak J. RNA-peptide fusions for the in vitro selection of peptides and proteins. Proc. Nat. Acad. Sci. USA 1997 Vol 94 pp. 12297-12302

Gold L. Polisky B., Uhlenbeck 0. & Yarus M. (1995) Ann. Rev. biochem. Vol 64 pp 763-797.

### BACKGROUND OF THE INVENTION

The single stranded RNA genomes of positive strand RNA bacteriophages such as Q-beta are replicated by means of an RNA replicase. The enzyme is produced in infected cells when a subunit encoded by the bacteriophage combines with subunits encoded by the bacterial cell to generate a multi-subunit replicase. This enzyme can use the positive strand from the infecting bacteriophage as a template for production of a complementary strand, the minus strand. Both the plus and minus strands can function as templates for the next round of replication. This leads to an exponential amplification of the bacteriophage genome. Although the enzymes exhibit a great deal of specificity for Bacteriophage RNA as templates for exponential amplification they can also catalyze the exponential replication of certain small RNA molecules. The prototype of such molecules is Midi-variant, usually referred to as MDV. This 221 nucleotide RNA molecule can function as a template for synthesis of it's complementary RNA strand by Q-beta replicase in the presence of ribo-nucleotides and magnesium in an appropriate pH and temperature range. Because the daughter strand so made can also function as an efficient template for synthesis of it's complement (the parent strand) the result is an exponential increase in the number of molecules of each strand in the mixture. One molecule of this template can give rise to a few micrograms of RNA in as little as 15 minutes under favorable reaction conditions.

In addition to MDV, other molecules are known which can function as templates for exponential amplification by q-beta replicase. These include, for example, MNV11, WS1, RQ120, RQ135 and others. Each of these molecules is a member of a family of closely related sequences. Several such families are known and others may remain to be discovered. Many mutated relatives of each replicator can function efficiently in amplification reactions. Under fixed conditions, one variant or another may have a greater ability to replicate. For example, a mutant MDV was selected which was better able to replicate in the presence of ethidium bromide than the parental molecule. Each of these molecules is referred to as a ‘replicator’.

This amplification phenomenon has been used in assays. In the simplest case replicator molecules containing hybridization probe sequences, inserted at a location which interferes minimally with replication, are used as probes in hybridization assays to detect complementary nucleic acid targets. Non-hybridized probe molecules are washed away and the remaining molecules are amplified to the point where they can be detected by conventional methods (eg by fluorescence in the presence of dyes) and interpreted as an indication of the presence of target molecules. Because of their high efficiency as templates every non-hybridized probe molecule must be washed away to eliminate background signal in assays of this type. This has led to elaborate washing schemes including repeated cycles of capture on and release from surfaces such as magnetic particles.

To avoid this washing requirement several schemes were devised such that the replicator probes would have reduced replicatability unless exposed to the correct target nucleic acid. One such scheme involved the attachment of inhibitory RNA sequences to the 3′ end of a replicatable molecule and their removal in a target dependent fashion by means of a ribozyme whose structure was completed by the presence of the target molecule. Another scheme was to use pieces of replicatable RNA, none of which were able to replicate without the others, and to ligate them together in a target dependent manner to produce the fully replicatable RNA molecule. Yet another scheme was to use fragments of DNA which are not independently replicatable and to ligate them together in a target dependent manner to produce a contiguous sequence which can be used as a template for production of fully replicatable RNA molecules.

There are limitations with all of these schemes, however. The inhibition of replication by adding 3′RNA extensions to the replicator is not very effective in practice so that in hybridization assays a very substantial washing requirement remains. The binary probe schemes involving ligation have two common problems:

1. The introduction of probe sequence into the replicator sequence both slows replication and increases the number of molecules required to get a response (ie sensitivity is reduced).

Because two probe fragments must hybridize with discrimination the probe sequence is typically longer than that required by the original unitary probe method. Long inserts in a replicator result in longer amplification times. The sensitivity reduction can be avoided to some extent by bounding the probe sequence with empirically chosen spacer sequences, which separate the probe from the replicator. Such spacer elements are identified empirically by a screening process. This is a laborious and time-consuming activity, which does not always result in structures with single molecule sensitivity. In addition, the spacers further increase the size of the insert and this also prolongs the amplification time.

2. The template activity of the replicator is greatly reduced by splitting it into two pieces, as done in the binary probe methods that have been described. However, the ability of the replicase, with very low efficiency, to use the 3′ fragment of a replicator as a template for replication can result in false positives in amplification assays. Typically if an amplification reaction contains more than about 10⁵ molecules of the 3′ fragment an amplification response will result. This places great demands on the washing technology. These demands have limited the application of the amplification technology.

A further problem can occur with some binary probe schemes. If the two pieces (the 5′ piece and the 3′ piece) of a binary probe replicator are mixed together at high concentration, a small fraction of the pieces can come together to form complexes capable of functioning as templates for the replicase. The formation of these “HOP complexes” does not require the presence of a hybridization target. Their formation can be inhibited by the addition of oligonucleotides complementary to short segments of one of the binary probe fragments (HOP blockers). This further complicates the assay and is not entirely successful since inhibition of HOP complex formation is not complete.

Strategically, these schemes start with an excellent replicator and try to reduce it's replicatability such that it can be restored in a target dependent manner. However, the attempts to implement this strategy are affected by three significant limitations:

1. Inactivation of replicator is incomplete as described above this results in assay background which imposes washing requirements and increases assay complexity.

2. When the assay requires that replicatability be restored in response to target the restoration of replicatability is incomplete (because of hybridization inefficiencies, washing effects and ligation limitations).

3. Furthermore, ligation restores, not the original efficient replicator, but the molecule with reduced replicatability that contains probe and spacer inserts.

### BRIEF SUMMARY OF THE INVENTION

This document describes a novel strategy for using Q-beta replicase in assays. This strategy provides certain advantages not found in other methods:

1. The probe is a chimera of RNA and DNA that does not include the complete sequence of a replicator. The 3′ termini needed for efficient initiation of replication are not present. The absence of the 3′ terminal sequences reduces assay background.

2. The nucleotide sequence of the probe molecule is such that the nucleic acid sequences that encode the replicator are permuted and inverted to eliminate replicatability. It is only by means of Reverse Transcriptase activity that the order of sequence elements and consequently the sequence of the replicator can be restored.

3. Although the probe molecule can contain a hybridization probe sequence for any target, the assay generates a complete replicator lacking inserts and which has the full replication ability of the original replicator.

Generally, there are three kinds of assays to which this strategy can contribute.

1. Reverse Transcriptase Assays

A method is described which makes the generation of efficient replicators completely dependent on the enzyme reverse transcriptase. This is an ultra-sensitive assay for reverse transcriptase activity. This is also the basis of the nucleic acid hybridization assays and the ligand-target assays described below.

2. Nucleic Acid Hybridization Assays

Methods are described which link the reverse transcriptase assay to nucleic acid hybridization assays, allowing Q-beta replicase to be used for ultra-sensitive detection of nucleic acid targets without the background signal exhibited by the other methods. Methods are described which make the Reverse Transcriptase facilitated generation of replicatable RNA dependent on the presence of hybridization targets. These are referred to as ‘smart probe’ methods.

3. Ligand Target Assays

A further elaboration of this method allows it to be used for detection of targets independent of nucleic acid hybridization. This involves the use of nucleic acid sequences, aptomers, which bind to target molecules without forming the double stranded structures typical of nucleic acid hybridization. For example RNA aptomers which bind a protein can be used for ultra-sensitive detection of that protein by Q-beta replicase. Ligands moieties may be chemically coupled to the chimeric molecules, preferably at the d-e insert. ‘Smart probe’ ligand-target assay methods are described.

The Reverse Transcriptase Assay

Reverse transcriptase (RT) is an enzyme which catalyses the condensation of deoxy-ribonucleotides to generate DNA with a deoxy-nucleotide sequence complementary to a template RNA molecule. The reaction requires an RNA template and a primer, which can be DNA or RNA. The product of reverse transcription is a DNA molecule which remains on it's RNA template in the form of a double stranded DNA-RNA hybrid. In the life-cycle of retroviruses the RNA strand is removed by RNAase H, an enzyme which hydrolyses the RNA strand of RNA-DNA hybrids.

The assay described here makes use of a chimeric molecule that includes an RNA segment preceded by a DNA segment and bounded on the other side by another DNA segment partially complementary to the RNA sequence and capable of functioning as a primer for reverse transcription. The template for reverse transcription is within the RNA segment that forms part of the tripartite chimeric molecule. After the reverse transcription reaction the template RNA segment is removed by digestion with RNAaseH. The RNAaseH activity results in the generation of two DNA molecules, the shorter of which primes the synthesis of DNA complementary to the longer molecule. The product of this reaction is a double stranded DNA molecule encoding the replicator nucleotide sequence downstream of a promoter for T7 RNA polymerase. Transcription by T7 RNA polymerase results in production of the non-interrupted replicator RNA that can be exponentially amplified by Q-beta replicase.

The general scheme, referred to as ‘the basic method’ is illustrated in the FIG. 1. FIG. 1 illustrates the basic scheme which can be used for assaying reverse transcriptase. The other two assay classes to which the invention applies involve inserting additional nucleic acid sequences ( called d/e inserts) between d and e in the chimeric molecule illustrated in FIG. 1. For nucleic acid hybridization assays the inserted sequence (the d/e insert) is complementary to a target sequence which is to be assayed. For ligand-target assays the inserted sequence contains a sequence which binds to the target being assayed. In this case the target might be a protein molecule and the d/e insert an RNA aptomer specific for that protein. In both of these assay classes modifications are described which reduce background signal from chimeric probe molecules which may be present but not bound to target. The d-e insert may not itself bind to a target analyte but may be chemically coupled to a moiety which can bind a target of interest-e.g. an antigen, biotin, antibody, streptavidin etc.

### BRIEF DESCRIPTION OF THE SEVERAL VIEWS OF THE DRAWINGS

FIG. 1a

Part I shows a chimeric probe molecule composed of a piece of RNA bounded at both ends by DNA segments. The thick line, from a to b represents the first DNA segment starting at the 5′ end. The RNA segment extending from c to d, is represented by the thin line. The RNA segment ends at d where it joins the second DNA segment which extends from e to it's 3′ end at g. Part of the second DNA segment, from f to g, is composed of a deoxy-nucleotide sequence complementary to part of the RNA segment to which it can hybridize so as to prime synthesis by reverse transcriptase.

Part II shows the same molecule as in part I but after reverse transcriptase has made a DNA copy of the RNA strand, starting at g where priming takes place and extending to the end of the template at h. The lower dotted line from g to h represents the DNA product of the reverse transcription reaction.

Part III illustrates the same molecule as Part II but after RNAaseH activity has digested the RNA strand of the double stranded region produced by copying the RNA strand as DNA. The top dashed line represents the degraded RNA.

Part IV shows the same molecule as part III but after the RNA strand has been completely digested. Note that this is a partially double stranded DNA molecule. The short segment corresponding to the first DNA segment, from a to b in part I above, is annealed to the 3′ end of the DNA strand generated by reverse transcription.

Part V illustrates the same thing as part IV.

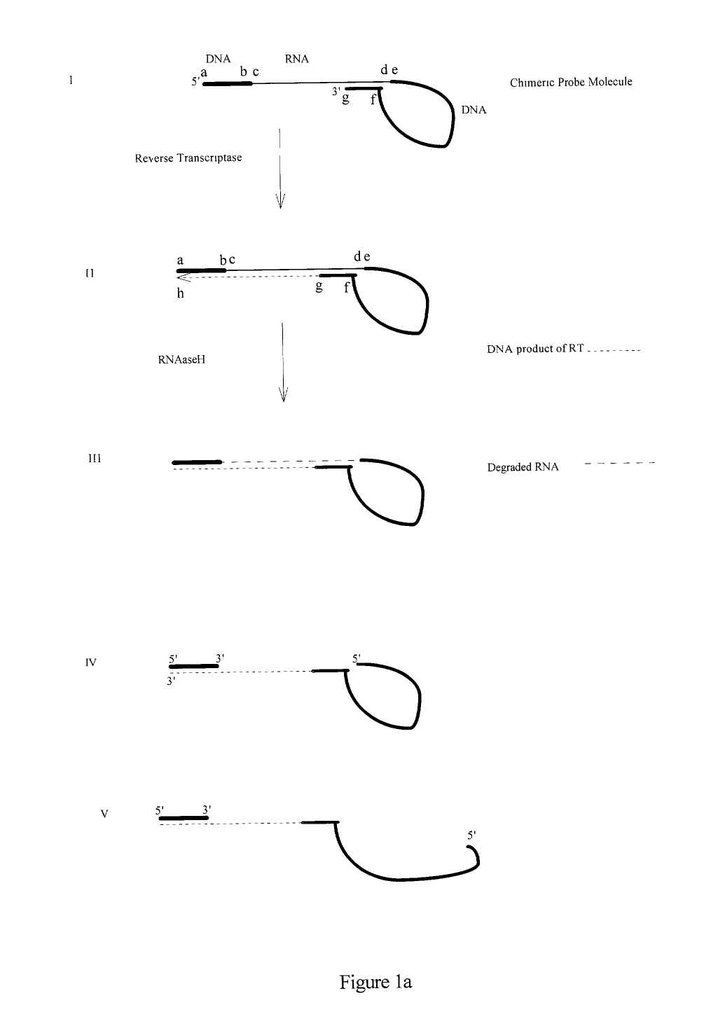

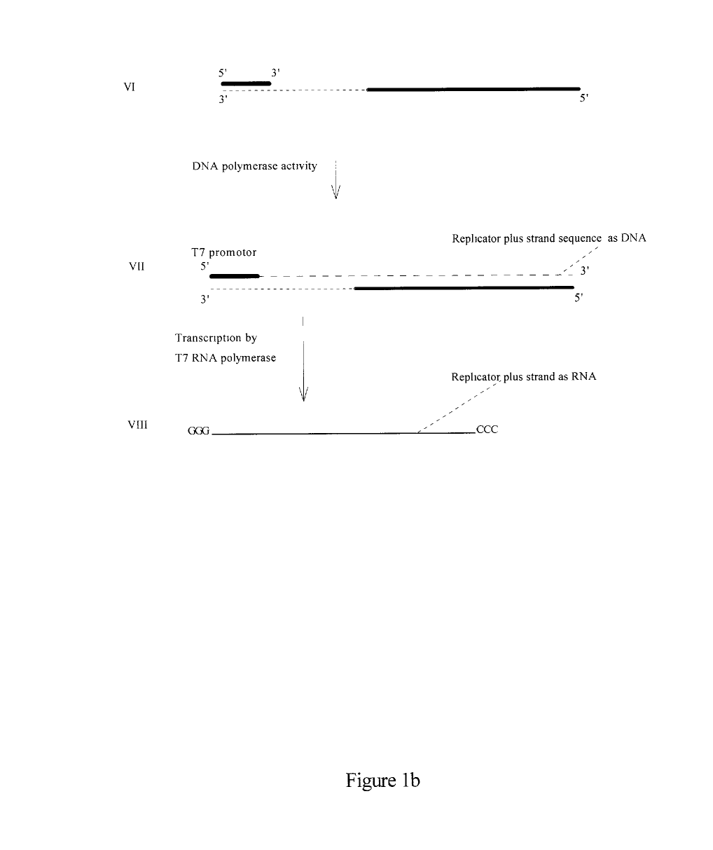

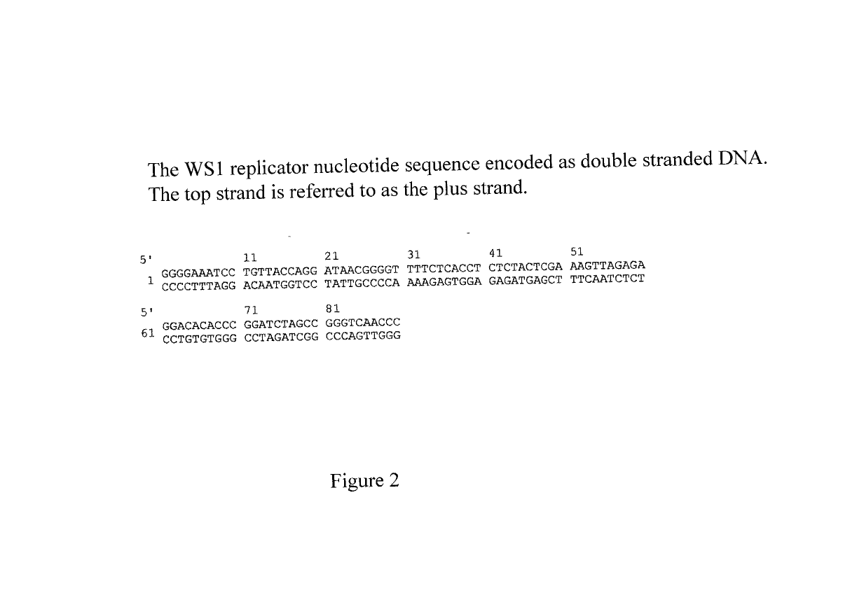

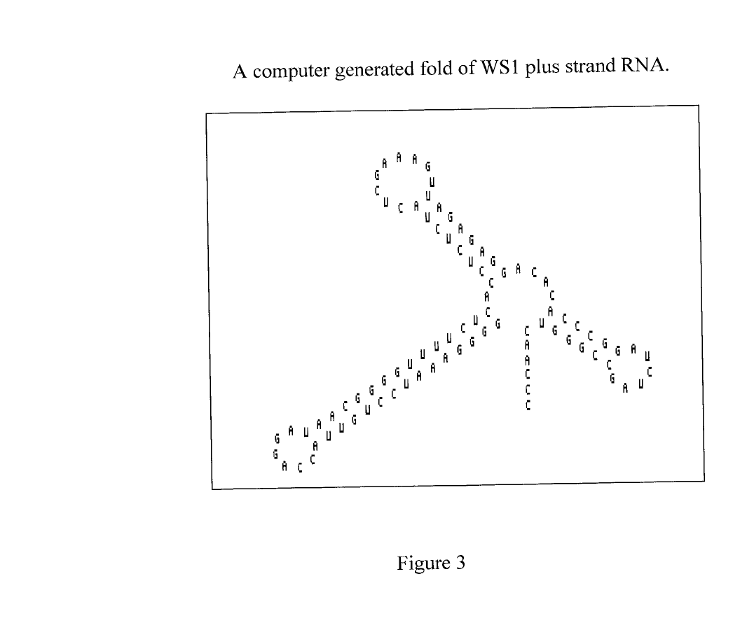

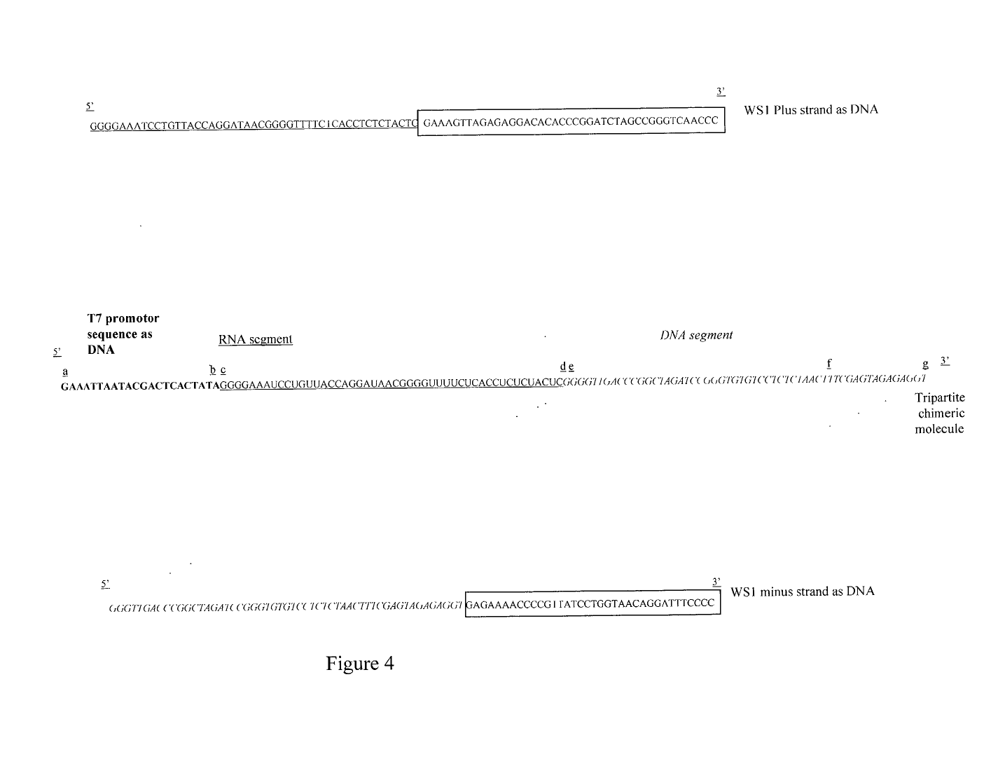

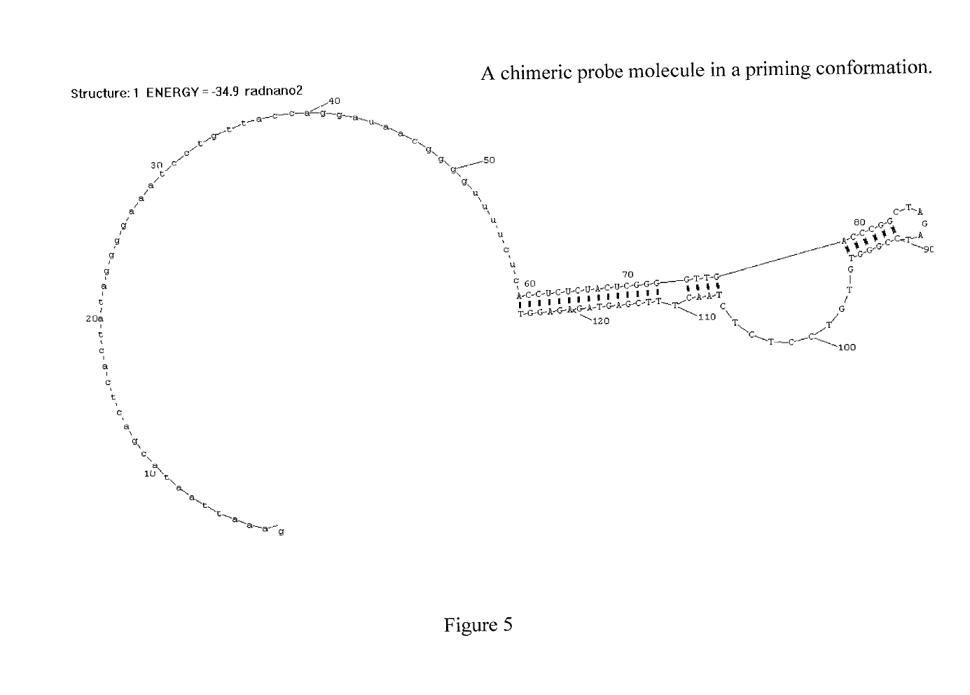

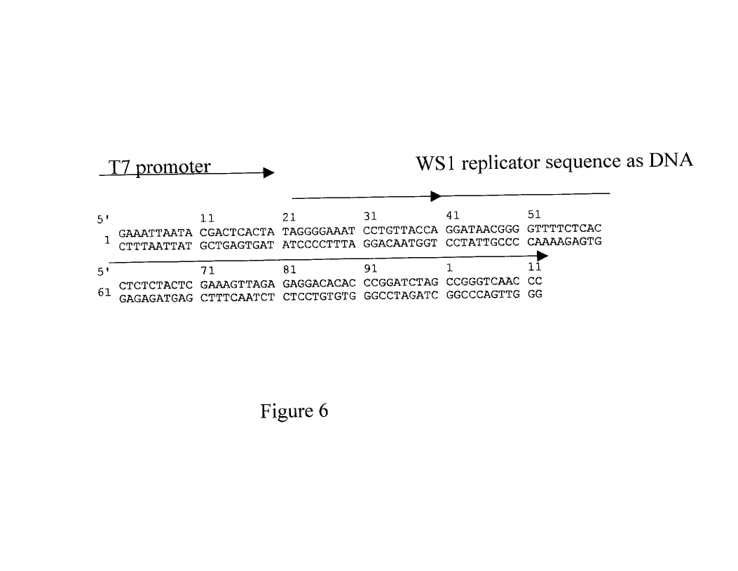

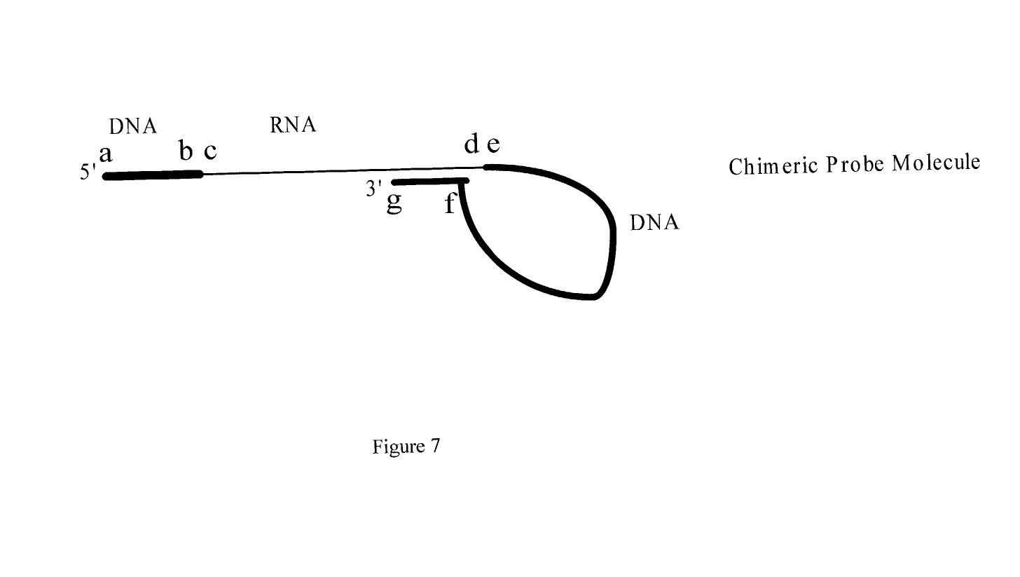

FIG. 1b

Part VI shows the same thing as FIG. 1 a part V but in a simpler drawing. This shows the primer-template combination resulting from the enzymatic activities described above. This is the structure on which DNA polymerase activity generates a double stranded DNA molecule.

Part VII shows the resulting double stranded DNA molecule. The top dashed line represents the newly made DNA. The DNA segment corresponding to the first DNA segment, a to b, in FIG. 1a part I is here identified as including a sequence which can function as a promoter for T7 RNA polymerase.

Part VIII illustrates the RNA made by transcribing the molecule shown in part VII with T7 RNA polymerase. This RNA begins with the 5′ GGG and ends with the CCC3′ OH sequence typical of the replicators described in the text. Such replicators are efficient amplification templates for Q-beta replicase. One molecule can rapidly be amplified to produce micrograms of RNA in a short time under appropriate reaction conditions.

FIG. 2

The nucleotide sequence of double stranded DNA encoding the replicator, WS 1. The top strand represents the plus strand of WS 1 as DNA.

FIG. 3

The RNA sequence of WS 1 plus strand; a computer generated folded structure of the RNA is shown.

FIG. 4

This shows three strands of nucleic acid. All three are shown with the 5′ end at the left and the 3′ OH end at the right, as indicated in the figure. The top strand shows the DNA representing the plus strand of the WS 1 replicator. This is the same as the top strand shown in FIG. 2. The bottom strand shows, as DNA, the minus strand of the WS 1 replicator. This is the same as the bottom strand shown in FIG. two but here the 5′ end is on the left. The middle section shows a chimeric molecule made up from part of the plus strand and part of the minus strand of WS 1 nucleotide sequence together with DNA corresponding to the top strand of a promoter for T7 RNA polymerase. The part of the WS 1 plus strand that is in the chimera is made of RNA. The part of the WS1 minus strand present in the replicator is made of DNA. The T7 RNA polymerase promoter segment is made of DNA. The result is a chimera consisting of and RNA segment bounded at each end by a DNA segment. The chimera is labeled with lower case letters, a through g, corresponding to the labelling in FIG. 1a and 1b. The first DNA segment, the T7 promoter, extends from a to b (bold letters). From c to d (underlined letters) is an RNA segment composed of the first 47 nucleotide residues of the plus strand of WS 1. From e to g (upper case italics) is a DNA segment corresponding to the first 54 nucleotides of the minus strand of WS 1.

FIG. 5

This shows the chimeric molecule from FIG. 4 and how it can form a structure capable of priming the reverse transcriptase reaction. The nucleotide residues are numbered from the 5′ end. (The use of lower case letters for part of the molecule is an illustration device required by the graphics program and has no other significance.)

FIG. 6

This shows the double stranded DNA resulting from the application of the enzymatic treatments described in the basic method to the tripartite chimeric molecule shown in FIGS. 4 and 5 above. The first 22 nucleotides comprise a T7 promotor for transcription beginning at nucleotide 23 and continuing to the end of the molecule. The transcript is a single stranded WS 1 plus strand RNA. This is an amplification template for Q-beta replicase.

FIG. 7

This is the same as FIG. 1a part I.

### DETAILED DESCRIPTION OF THE INVENTION

A replicator called WS 1, has been chosen for demonstrating this invention. In addition to being an excellent replicator, WS1 is only 90 nucleotides long and it's small size facilitates the synthesis of the chimeric probe molecules which are the subject of this invention. The nucleotide sequence of double stranded DNA encoding the WS 1 RNA is shown in FIG. 2. The top strand is referred to as the plus strand of WS 1. A computer generated folded structure of the 90 nucleotide WS 1 plus strand RNA is shown in FIG. 3. An example chimeric molecule, showing how parts of the WS 1 nucleotide sequence are incorporated into a tripartite chimera and identifying the boundaries of the RNA and DNA segments is shown in FIG. 4 and FIG. 5.

In the 125 nucleotide chimeric probe molecule shown in FIG. 4 and FIG. 5, there are three segments.

1. Residues 1 to 22 are deoxy-ribonucleotides, the sequence of which, correspond to one strand of a promoter for T7 RNA polymerase.

2. Residues 23 to 70 are ribonucleotides in the sequence of residues 1 to 47 of the plus strand of WS 1.

3. Residues 71 to 125 are deoxyribonucleotides corresponding to the sequence of nucleotides 1 to 54 of the minus strand of WS 1. Neither residues 48 to 90 of the plus strand nor residues 55 to 90 of the minus strand are present in the chimera. This chimera contains self-complementary sequences, which form stem-loops and duplex regions, some of which are shown in the computer generated structure in FIG. 5. Note the duplex formed by the annealing of residues 59 through 71 to the complementary sequence extending from residue 1 11 to residue 125.

This duplex will be referred to as the priming segment.

The proximal part of the primer segment (residues 59 through 71) will be called the primer binding sequence (PBS).

The distal part (residues 111 to 125) will be called the primer.

The segment preceding the PBS, residues 1 to 22 (DNA) through residues 23 to 70 (RNA) is called the RT template.

The PBS is composed of ribonucleotides (RNA). The Primer is composed of deoxyribonucleotides (DNA).

The RT template is composed of both RNA and DNA as indicated in FIG. 3 and FIG. 4.

Reverse transcription of this template, initiated on the primer, produces DNA complementary to residues from 58 back to residue 1. The chimera is elongated from the primer by the addition of deoxy-ribonucleotides starting with residue 125 and continuing to residue 184. This activity generates, as DNA, both the 3′ end of the minus strand of WS 1 and the bottom strand of the T7 promoter, all as parts of the resulting 184 nucleotide long chimeric molecule.

RNAaseH treatment of the resulting molecule digests the RNA segment, residues 23 to 70, allowing residues 1 to 22 (DNA) to function as a primer for DNA synthesis. Synthesis primed from this site uses the DNA made by reverse transcription as the initial part of the template and the contiguous residues 153 to 76 as the distal portion of the template.

The result is a double stranded DNA molecule from which T7 RNA polymerase can transcribe the complete WS 1 replicator. This is shown in FIG. 6. The addition of Q-beta replicase and ribonucleotide triphosphates under appropriate buffer and temperature conditions causes RNA amplification, resulting in accumulation of RNA to a point where it can be detected by routine methods.

Further Development of the Invention

Consider the general chimeric probe molecule illustrated in FIG. 1a , part I, and shown below. This is composed of three segments. From a to b is a DNA segment which encodes the top strand of a T7 RNA polymerase promoter and, perhaps, the 5′ end of the replicator. From b to e is composed of RNA and encodes part of the plus strand of a replicator. This b to e segment would include the 5′ end of the replicator if this is not encoded in the a to b segment. The entire segment from b through c to d is in the contiguous sequence of the replicator (partly as DNA and partly as RNA) from it's 5′ end to an internal position chosen on the basis of considerations outlined below. The third segment extending from e to g is composed of DNA and encodes part of the minus strand of a replicator, including the 5′ end which begins at e.

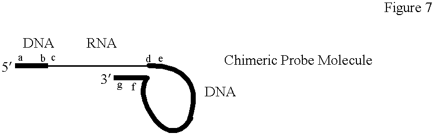

Certain points about these segments are highlighted here

1. The T7 promoter segment encodes only one strand of the promoter and is not, without it's complement, a functional promoter. Furthermnore there is no template strand from which it might promote transcription.

2. The RNA segment, c to d, encodes, as RNA, part of the plus strand of the replicator. This may include the 5′ end or it may in some cases be preferable to encode the 5′ end as DNA to reduce replicatability. The choice of how much of the plus strand of the replicator sequence is present in the c to d segment is based on these considerations:

a. Since it is RNA and therefore inherently a better potential Q-beta replicase template than the DNA segments, it should be as short as possible. It must nevertheless be long enough to anneal to the priming segment.

b. Also, since it is RNA, it should lack high affinity binding sites for Q-beta replicase. Such high affinity binding sites typically occur in good replicators. A good strategy is to chose strands and breakpoints which permit the nucleotide sequences that (as RNA) comprise the high affinity binding sites for Q-beta replicase to be located in DNA segments.

3. The RNA segment, c to d, must be long enough so that when it is in the form of an RNA/DNA hybrid, as after reverse transcription, it is a good substrate for RNAaseH. It should be at least 8 nucleotides long but can be much longer.

4. The segment e to g0 is DNA comprising part of the nucleotide sequence of the minus strand of the replicator. It must contain the primer segment, f to g and end with a 3′ hydroxyl, capable of priming reverse transcription. The segment e to g includes, as DNA beginning at e, the 5′ end of the minus strand of the replicator nucleotide sequence.

5. The segment f to g must be complementary to the 3′ end of the RNA segment c to d as shown in the figure. This segment must be long enough to form a stable priming duplex with it's complementary sequence in the c to d segment as shown. The priming DNA segment, f to g, must be long enough so that it's stability forces a priming configuration on the chimeric molecule. It must be chosen and tested to ensure that this occurs.

Variations on the Theme

1. In the general scheme described above the RNAaseH reaction results in a long DNA molecule with a shorter molecule annealed to it. This structure can prime DNA synthesis catalyzed by a DNA polymerase to generate the double stranded DNA molecule from which the RNA replicator can be transcribed by T7 RNA polymerase. However, the DNA polymerase activity is not necessary for the assay because T7 RNA polymerase can use the RNAaseH product as a template for efficient transcription of the replicator. This is possible because reverse transcriptase activity has already generated the complete T7 promoter and the transcription template. Although it's promoter is double stranded DNA,T7 RNA polymerase does not require that the template strand be part of a double stranded structure. This can eliminate a step in the assay. Only three enzymatic reactions are then required to produce the molecules which can be amplified by Q-beta replicase with single molecule sensitivity. Reverse transcriptasae, RNAaseH, and T7 RNA polymerase. In some cases, however, extensive secondary structure in the template strand could interfere with transcription of a single stranded template by T7 RMA polymerase. Converting the RNAaseH product to the double stranded DNA structure shown in the general scheme can help to overcome such interference and double stranded DNA is the preferred template of T7 RNA polymerase (REFS).

2. The DNA segment a to b, encoding the T7 promoter, can be omitted from the chimeric probe molecule. This permits a bipartite chimeric molecule to be used in place of the tripartite chimera shown in the general scheme. This bipartite chimera includes the RNA segment c to d (which would include the 5′ end of the replicator) and the DNA segment e to g. Using such a bipartite chimeric probe the result of reverse transcription and RNAase H activity is a single stranded DNA molecule comprising, as DNA, the complete minus strand of the replicator. Such DNA molecules can function as templates for generation of RNA by Q-beta replicase. Typically, for a replicator that gives a response from one molecule of RNA a response from the corresponding DNA requires about 100 DNA molecules. This can provide adequate sensitivity in some applications and would permit the use of a bipartite chimera rather than a tripartite chimeric molecule. From a manufacturing point of view this would have value. This variation requires only two enzyme reactions prior to Q-beta replicase amplification, reverse transcription and the RNAaseH reaction. The sensitivity of such an assay can be further increased, by using the promoter-independent ability of T7 RNA polymerase to generate RNA from single stranded DNA templates. A small fraction of the transcripts initiated on single stranded DNA proceed to the point where the enzyme takes on it's elongating conformation and continues transcribing to the end of the template. Each transcript molecule generated in this way is a template for Q-beta replicase.

d/e Inserts

d/e inserts are the basis of both nucleic acid hybridization assays and ligand-target assays using the chimeric molecules described here.

In FIG. 7, taken from FIG. 2, which illustrates the basic method, position d represents the last ribonucleotide before the second DNA segment begins. In the basic method this is a part of the replicator sequence selected as described above. Position e is the first deoxynucleotide residue after the RNA segment and is, as DNA, the first nucleotide of the minus strand of the replicator-usually the first of several deoxy-guanosine residues. As shown in the general scheme, ribonucleotide residues preceding this do not occur in the template generated by the RNAaseH activity that occurs after reverse transcription. (Additional nucleotides in the initial Q-beta replicase template molecule can have a small affect on initiation of the first round of amplification by Q-beta replicase but they do not occur in the product of that round or interfere with subsequent amplification. Nevertheless it is preferable to exclude them.)

It is possible to insert additional nucleotide residues between d and e without disrupting the structure of the replicator eventually produced by the assay because such additional residues are external to the replicator coding sequence to which they become appended at the 3′ end. These will be referred to as d/e inserts.

Nucleic Acid Probes

One use for d/e inserts lies in their application as hybridization probes. A ribonucleotide sequence complementary to a target sequence can be inserted between d and e. The resulting chimeric molecule can be used as a hybridization probe. In the simplest case the chimera can be hybridized to target nucleic acid, the non hybridized molecules washed away and the residual molecules subjected to reverse transcription, RNAaseH treatment, optional DNA polymerase treatment, transcription by T7 RNA polymerase and amplification by Q-beta replicase. However this assay would require a very effective washing method because every probe molecule has the potential to become a replicator when subjected to the enzyme treatments described in the ‘basic method’.

‘Smart Probe’ Methods for the Reduction of Assay Background are Described Here

1. Background Reduction by Chain Termination

Generally, the hybridization of d/e inserts to target nucleic acid would inhibit the priming DNA sequence, f to g, from annealing to it's complement, the primer binding sequence located in the RNA segment, c to d. Target sequences can be chosen such that their complements maximize this inhibition. Longer probe sequences are more effective than shorter sequences. Shorter primer sequences are more subject to inhibition than their longer counterparts. By bounding the probe sequence with additional spacer elements, empirically chosen, it is possible to further maximize the inhibition of priming in a probe-target complex.

After hybridization of such a chimeric probe to target nucleic acid the result is typically a mixture of hybridized and non-hybridized probe molecules. The non-hybridized molecules are capable of priming reverse transcriptase activity. The hybridized probes are in a conformation that prevents this priming. The addition of chain terminating nucleotides (e.g. dideoxy nucleotide tri-phosphates, ddNTP's) and reverse transcriptase at this point results in the incorporation of the ddNTP into any molecules that can function in the priming reaction, thereby terminating their priming ability. The hybridized probe molecules are unaffected by this reaction. The subsequent addition of an excess of dNTP's and release of the probe from it's target allow the molecules which had been hybridized to recover their priming ability. This is most easily envisioned by considering an assay format usually referred to as a ‘sandwich assay’ in which the probe-target complex is captured (e.g. by a biotinylated oligonucleotide) on a surface (e.g. a magnetic particle coated with streptavidin). The initial Reverse Transcriptase activity in the presence of chain terminating nucleotides can be done while the probe is on the surface. Washing to remove the chain terminating nucleotides is followed by release of the hybridized probes from the target followed by reverse transcription in the presence of dNTP's and continuing the assay as described above.

In some cases a homogeneous assay may be preferred. This could be done by performing the terminating reaction with a low concentration of chain terminating nucleotides (e.g. a ddNTP) and then using a vastly greater amount of dNTP for the second RT reaction which takes place after release of probes from target. Since this application is not an assay for RT, the nature of the particular RT being used can be chosen to facilitate the assay. For example the termination step could be done using an RT that efficiently incorporates chain terminating nucleotides while the second step could be done, possibly after inactivating the first RT, using an RT that preferentially incorporates dNTP's even in the presence of chain terminating nucleotides. Such enzymes may occur naturally or be generated by mutational methods.

2. Background Reduction by RNAase H

A unitary ‘smart probe’ method is also possible using RNAaseH. A background reduction step would be a post-hybridization treatment with RNAaseH. Non-hybridized unitary probes form priming competent complexes. These are substrates for RNAaseH activity which can destroy the primer binding segment (complementary to f-g). Subsequent inactivation or removal of RNAaseH followed by release of hybridized probe molecules from target is followed by the basic method as described above (FIG. 2). Such background reduction steps reduce washing requirements and facilitate simple assay protocols and assay automation

3. Background Reduction by Using a Binary Chimera

A second way to make the generation of replicator molecules dependent on hybridization to target molecules involves making the chimeric probe in two pieces, each containing part of the hybridization probe segment. In this case a chimeric probe is designed by placing a probe sequence as a d/e insert. A breakpoint is chosen within the d/e hybridizing segment. The chimeric probe molecule is then produced as two separate bipartite chimeric pieces which, when ligated together, generate the tripartite chimera. When the two segments are hybridized to a target molecule the ends are juxtaposed so as to permit priming without ligation or ligation to generate the molecules which can form a priming structure. In the latter case hybridization to target is followed by ligation to generate the chimeric molecules from which replicator template can be generated by reverse transcription, RNAaseH, DNA polymerase, and T7 RNA polymerase activities as described above. In this case ligation is done either enzymatically using T4 DNA ligase or by chemical methods. Non-hybridized probe molecules are not brought together and consequently not ligated. Neither of the two individual chimeric probe segments can be RT templates because each lacks either the priming segment or the primer binding segment. Furthermore, if aberrant priming of RT does occur within one of the binary probe fragments, this does not result in the generation of a replicator. For some probe designs, hybridized, ligated molecules, although they cannot prime RT activity because of conformational constraints, contain all the required sequences. Neither of the free pieces encode a complete replicator sequence and both of them lack the 3′ ends needed for initiation of replication.

4. d/e Insert as RNA

The d/e insert comprising the hybridization probe can be either RNA or DNA. If it is RNA then no further assay modifications are required. If it is DNA then a further consideration becomes relevant. According to the basic method shown in FIG. 2 the nucleotide sequence comprising the d/e insert becomes an extra-replicator segment contiguous with the 5′ end of the minus strand of the replicator which is generated as DNA by the combined activities of RT and RNAaseH. If the hybridization probe sequence is made as RNA then this extra segment is also composed of RNA. In the standard scheme this segment becomes part of the template for synthesis of the plus strand DNA by the second round of RT activity which results in it being part of the resulting double stranded molecule. This makes it subject to the activity of RNAaseH which removes it. The resulting template strand contains only non-interrupted replicator sequence without additions.

5. d/e Insert as DNA

However, if the d/e insert is composed of DNA then the extra-replicator sequence becomes part of the double stranded transcription template as described above but, being DNA, cannot be removed by RNAaseH. In this case the sequence of the d/e insert at the junction with the 5′ end of the minus strand should be chosen such that a restriction endonuclease can remove the extra sequence from the resulting double stranded DNA molecule. A good choice of sequence is CCCGGG which is cleaved by SmaI between the middle C and G. In this case the first G in the sequence is the 5′ G of the minus strand of the replicator which typically begins with GGG. The d/e insert must end with CCC which will be juxtaposed to the GGG at e to generate the SmaI site. Removal of the extra-replicator sequence is advantageous because of a small effect on sensitivity in Q-beta replicase amplification caused by such additions.

6. Background Reduction by Combining Binary and Chain Termination Methods

Some assay background could occur due to the two probe fragments inefficiently coming together without target. This could produce a priming-competent configuration. Therefore a further background reduction step may be advantageous. A further elaboration of the method is to use the binary method described above followed by the chain terminator method, described in 1. above, for additional background reduction if needed.

7. Background Reduction by Blocking Oligonucleotides

An alternative additional background reduction step is the addition to the assay of short oligonucleotides which interfere with target independent annealing of the two binary probe fragments to inhibit formation of priming-competent complexes. The length, nucleotide sequence and composition of these molecules can be chosen based on empirical studies depending on the replicator being used. The parameters have to be chosen such that annealing of the binary probe fragments to target is not unduly inhibited. These primer-blocking oligonucleotides would lack the 3′ hydroxyl group needed for RT priming activity. This step could be combined with the RNAaseH based background reduction steps decribed below. Nucleic acid analogs could be used for this blocking function e.g PNA.

8. Background Reduction by RNAaseH in Binary Method

Another alternative background reduction step would be a post-hybridization treatment with RNAaseH. Any non-hybridized binary probe fragments which come together to form priming-competent complexes will become substrates for RNAaseH activity. This will destroy the primer binding segment (complementary to f-g) eliminating the possibility of subsequent RT activity on these molecules and removing a vital part of the replicator sequence. Inactivation or removal of RNAaseH followed by release of hybridized probe molecules from target is followed by the basic method as described above (FIG. 2). Such background reduction steps reduce washing requirements and facilitate simple assay protocols and assay automation. Since this approach does not result in probe sequence within a replicator the amplification step does not place constraints on the choice of probe sequence or length. The hybridization requirements can therefore be given greater weight in the choice of probe sequence and length.

Ligand-Target Assays

Above I have described the use of chimeric DNA-RNA-DNA molecules for Reverse Transcriptase assays and similar molecules containing d/e inserts, composed of either RNA or DNA, for use in hybridization probe assays. A further application of d/e inserts is described here. Since d/e inserts are absent from the replicator generated by the assay they do not interfere with it's replication. This provides a wide latitude for the choice of such inserts.

The work of Szostack, and that of Gold and others (refs) has shown that selection procedures combined with combinatorial methods can be used to identify nucleic acid sequences which bind with high affinity to other substances. Such nucleic acids, here referred to as aptomers, can be composed of either RNA or DNA.

Here I describe a method for using such sequences, as part of chimeric molecules, for the ultra-sensitive detection of their cognate ligands. In essence the ligand-binding nucleic acid sequence is inserted into the basic assay chimera as a d/e insert. A binary version of the method makes use of two ligand binding segments chosen such that they can be ligated together when bound to target. Ligation, either chemically or enzymatically using for example T4 RNA ligase or T4 DNA ligase, generates a chimera from which a replicator can be produced by application of the basic method described above.

An RNA sequence that binds a certain ligand can be identified by the SELEX procedure, for example. In the simplest case this RNA sequence is used directly as a d/e insert, producing a chimeric probe molecule containing all the elements described in the basic assay in addition to the ligand-binding RNA segment. In a simple assay the chimeric probe is exposed under appropriate binding conditions to the ligand-containing sample, possibly fixed to a surface. After the binding reaction, non-bound probe molecules are washed away and the remaining molecules are used as templates for RT and the other basic reaction components to generate Replicator molecules which are then amplified by Q-beta replicase.

Further Elaborations of the Invention

1. Ligand-Target Assay Using Unitary Probe and Background Reduction by Chain Terminating Method or RNAaseH Method

Usually, the ligand-binding RNA sequence (aptomer) is originally identified in a nucleic acid sequence context different from that of the chimeric molecules described here. In some cases incorporation into these chimeras may interfere with ligand-binding activity of the aptomer. This inhibition can be prevented by separating the aptomer from the basic chimera by bounding it on one or both sides with spacer elements. The spacer elements may reconstitute the original sequence context in which the aptomer was identified or they may be chosen in the context of this assay based on empirical studies. They must not interfere with RT primer activity of non-bound molecules. If the aptomer-spacer combination results in inhibition of primer binding (and therefore reverse transcriptase activity) on ligand-bound probe molecules but not on free probe molecules then one or more of the background reduction techniques described above for hybridization assays can be used. Such aptomer-spacer sets can be found by a combination of design and empirical studies. After the ligand binding step the free molecules are reacted with chain terminating agents or RNAaseH or both. After removal of RNAaseH and chain terminators the bound chimeras are released and subjected to the steps of the basic method and amplified. This is a unitary probe method.

2. Binary Probe Methods Using Aptomers

In this case two aptomers are used, each being part of a separate piece of a chimeric binary probe. Conceptually, two aptomers are joined together and used as a d/e insert. For assays, the chimeric molecule is produced in two pieces, each containing one of the two aptomers. The chimera is split between the two aptomers. In this case the spacer considerations described above for a unitary aptomer-containing probe apply. There are additional spacer considerations here, however:

The assay requires that when the two aptomer-containing probe fragments are bound to the ligand they can either form a priming competent structure or be ligated together to produce the chimera which becomes the RT template from which the basic assay can produce replicator RNA for amplification by Q-beta replicase. This means that both aptomers, when part of separate molecules, must be able to bind simultaneously to the same ligand molecule and, when so bound, have ends in a conformation that allows them to form a priming competent structure or to be ligated together. Such aptomer combinations, and the sequences that separate them in which ligation occurs, can be chosen based on selection experiments and on empirical studies for each target species. Ligation can be done by T4 RNA ligase which ligates single stranded nucleic acids. T4 DNA ligase can be used if the aptomer separator element permits annealing to a short oligonucleotide which would produce a double stranded structure across the ligation point on ligand bound but not on free probe fragments. Chemical ligation is possible in either case. If RNA ligase is to be used then aptomer-spacer combinations can be designed such that the juxtaposition of the two probe fragments when bound to ligand would result in formation of an optimal structure for RNA ligation by this enzyme (ref Orgel). Additional background reduction using RNAaseH and/or chain termination methods may be added as described above for unitary ligand-target and for nucleic acid hybridization assays.

3. The use of aptomers with the chimeric molecules described here has another application in reverse transcriptase assays. For the reverse transcriptase assay it may be useful in some applications to use as a d/e insert an RNA sequence that functions as a high affinity ligand for a particular reverse transcriptase. This could allow the assay to preferentially detect that reverse transcriptase rather than others which could be present in the sample. For example RNA sequences that bind with high affinity to the HIV reverse transcriptase have been described. Such a sequence could be inserted between d and e. To avoid interference with the binding activity of this RNA to HIV reverse transcriptase empirically chosen spacer elements bounding the binding segment on one or both sides may be needed as described above.

4. Above, RT aptomers are used to increase the affinity of a chimeric probe for RT, thereby giving the assay increased specificity for that RT rather than others. The converse is also possible. Probes containing aptomers that inhibit one RT but not another can be used to assay other RT's in the presence of the one being inhibited. RNA aptomers which inhibit the HIV reverse transcriptase have been described (ref Brown and Gold.). Aptomers specific for other RT's are also described in the literature.(Gold.)

What I claim as my invention is:

1. The use in assays of chimeric RNA-DNA molecules, which do not directly encode a contiguous RNA sequence that can be exponentially replicated by Q-beta replicase, but which can form a structure that permits Reverse Transcriptase priming to generate DNA which does so encode complete contiguous RNA replicator molecules which can be subsequently transcribed by an RNA polymerase and exponentially replicated by Q-beta replicase, the Chimera being composed of
a. an RNA segment that corresponds to the 5′ end of the plus strand of the replicator appended at it's 3′ end to the 5′ end of a DNA segment that is the complement of the 3′ end of the replicator, the DNA segment being long enough to include deoxy ribonucleotide sequence complementary to part of the RNA segment which precedes it in the same molecule so that a structure can form which can function as a primer for reverse transcriptase, neither the nucleotide sequence that comprises the the 3′ end of the plus strand of a replicator nor the nucleotide sequence that comprises the 3′ end of the minus strand of a replicator being present as either RNA or DNA in the chimeric molecule but these sequences being encoded by the elements which are present;
b. a DNA segment preceding the RNA segment, the DNA being complementary to the ‘top’ strand of a promoter for an RNA polymerase (such as the bacteriophage T7 RNA polymerase) such that Reverse transcriptase activity generates both a double stranded promoter for the RNA polymerase and a primer for DNA synthesis which can generate a double stranded DNA molecule from which a complete replicator RNA molecule can be transcribed.

2. The use in assays of such chimeric RNA-DNA molecules for the detection of reverse transcriptase activities by virtue of their ability to give rise to replicator RNA molecules as described in claim 1a bove, the signal for the presence of reverse transcriptase being the products of RNA amplification by Q-beta replicase.

3. The use in assays of chimeric molecules described in claim 1 above but modified by the introduction of additional nucleic acid sequence, located within the chimeric molecules, described in claim I above, between the 3′ end of the RNA segment that corresponds to the 5′ end of the plus strand of the replicator and the DNA segment that is the complement of the 3′ end of the same replicator strand, such sequences being referred to as d-e inserts, not becoming part of the replicator generated by the procedures here described.

4. The use in assays of molecules described in claim 3 above in which the additional sequence (the d-e insert) can function as a hybridization probe for the detection of nucleic acids, the nucleotide sequence of the d-e insert being chosen so that part or all of it is complementary to a target nucleic acid of interest.

5. The use in assays of binary probe molecules in which each member of a binary pair includes a nucleic acid sequence complementary to a target nucleic acid being assayed, a binary pair being conceptually identical to a molecule described in claim 4 above which has been split within the d-e insert such that part of the hybridization probe is on each of the two resulting molecules so that their annealing to a target nucleic acid can, depending on details of the d-e insert and the structure of the target either:
a. juxtapose the ends so as to permit the two members to be ligated together with T4 DNA ligase or chemically so that the resulting ligated molecule may be able to form an RT priming structure either while still bound to the target or after release from the target nucleic acid so that Reverse Transcriptase activity can generate DNA encoding complete contiguous RNA replicator molecules which can be subsequently transcribed by an RNA polymerase and exponentially replicated by Q-beta replicase;
b. juxtapose the ends so as to permit the two members to be ligated together but prevent formation of a priming structure unless the resulting chimera is released from the target nucleic acid permitting priming competent molecules, which are not annealed to target nucleic acid but which may be present, to be inactivated by enzymatic incorporation of chain terminating chemicals so that when the annealed molecules are released from target they can form a priming structure which permits Reverse Transcriptase activity to generate DNA encoding complete contiguous RNA replicator molecules which can be subsequently transcribed by an RNA polymerase and exponentially replicated by Q-beta replicase;
c. juxtapose the two members so as to permit formation of a priming structure without ligation so that RT activity can generate DNA encoding complete contiguous RNA replicator molecules which can be subsequently transcribed by an RNA polymerase and exponentially replicated by Q-beta replicase.

6. The use in assays of molecules as described in 3. above in which the additional sequence ( the d-e insert ) includes or is chemically coupled to a structure which can function as a ligand that binds to non-nucleic acid target analytes (-e.g. proteins) the ligand being either:
a. a nucleic acid segment contiguous with the replicator sequences in the chimera, such nucleic acid sequences being either naturally occurring or artificial such as products of SELEX or other methods for selection of sequences from combinatorial libraries;
b. a chemically coupled ligand which binds directly to target analyte such a ligand being of natural or artificial origin including for example a molecule such as biotin or an antigen or other small molecule capable of binding to a target of interest or a peptide or a protein molecule such as an antibody, a lectin or streptavidin, or an RNA or DNA aptomer or a peptide product of phage display, ribosome display or mRNA display or of combinatorial chemistry, chemically linked to the chimera.

7. The use in assays of binary probe molecules similar to those described in claim 5 above but instead of hybridization probe sequence, each member of a binary pair includes or is chemically coupled to a structure which can function as a ligand that binds to non-nucleic acid target analytes (-e.g. proteins) the ligand being either:
a. a nucleic acid segment contiguous with the replicator sequences in the chimera, such nucleic acid sequences being either naturally occurring or artificial such as products of SELEX or other methods for selection of sequences from combinatorial libraries;
b. a chemically coupled ligand which binds directly to target analyte such a ligand being of natural or artificial origin including for example a molecule such as biotin or an antigen or other small molecule capable of binding to a target of interest or a peptide or a protein molecule such as an antibody, a lectin or streptavidin, or an RNA or DNA aptomer or a peptide product of phage display, ribosome display or mRNA display or of combinatorial chemistry, chemically linked to the chimera;
so that their binding to a target analyte can, depending on details of the d-e insert and the structure of the target either:
c. juxtapose the ends so as to permit the two members to be ligated together with T4 DNA ligase or chemically so that the resulting ligated molecule may be able to form an RT priming structure either while still bound to the target or after release from the target so that Reverse Transcriptase activity can generate DNA encoding complete contiguous RNA replicator molecules which can be subsequently transcribed by an RNA polymerase and exponentially replicated by Q-beta replicase;
d. juxtapose the ends so as to permit the two members to be ligated together but prevent formation of a priming structure unless the resulting chimera is released from the target nucleic acid permitting priming competent molecules, which are not annealed to target but which may be present, to be inactivated by enzymatic incorporation of chain terminating chemicals so that when the annealed molecules are released from target they can form a priming structure which permits Reverse Transcriptase activity to generate DNA encoding complete contiguous RNA replicator molecules which can be subsequently transcribed by an RNA polymerase and exponentially replicated by Q-beta replicase;
e. juxtapose the two members so as to permit formation of a priming structure without ligation so that RT activity can generate DNA encoding complete contiguous RNA replicator molecules which can be subsequently transcribed by an RNA polymerase and exponentially replicated by Q-beta replicase.

8. The use in assays of molecules as described in claim 7 above but in which each member of a binary pair has a different ligand coupled such that both ligands bind to the same target analyte, bringing the two pieces together to permit formation of a Reverse Transcriptase priming structure which can be used to generate complete contiguous replicator molecules which can be subsequently exponentially replicated by Q-beta replicase.

9. The use in assays of molecules as described in claim 7 above but in which each piece has the same ligand coupled such that both ligands bind to the same target molecule bringing the two pieces together to permit formation of a Reverse Transcriptase priming structure which can be used to generate complete contiguous replicator molecules which can be subsequently exponentially replicated by Q-beta replicase.
